SEQUENCE LISTING (2 sequences)

SEQ ID NO: 1 - DNA, 25 bp, synthetic construct
  source 1..25: mol_type = other DNA
tccgggagggggggtgggtggacgg

SEQ ID NO: 2 - DNA, 25 bp, synthetic construct
  source 1..25: mol_type = other DNA
ggtggtggcggggggtgggggggtt

(19) United States
(12) Patent Application Publication
(10) Pub. No.: US 2023/0330277 A1
(43) Pub. Date: Oct. 19, 2023

(54) Radioisotope and Aptamer Drug Combination to treat S. aureus and E. coli infections

(71) Applicant: eNano Health Limited, Hong Kong SAR (HK)

(72) Inventor: Patrick Shau-park Leung, Arcadia, CA (US)

(21) Appl. No.: 17/933,525

(22) Filed: Sep. 20, 2022

Related U.S. Application Data
(63) Continuation-in-part of application No. 15/469,138, filed on Mar. 24, 2017, now Pat. No. 10,513,725.
Application No. 15/469,138 is a continuation-in-part of application No. 15/056,163, filed on Feb. 29, 2016, now Pat. No. 10,506,954.
Application No. 15/056,163 is a continuation-in-part of application No. 16/374,838, filed on Apr. 4, 2019.

(51) Int. Cl. A61K 51/04; C12N 15/115; A61P 31/04

(52) U.S. Cl. CPC A61K 51/0491; C12N 15/115; A61P 31/04; C12N 2310/16

(57) ABSTRACT

The claimed invention provides a novel system and related method for treatment of bacterial infections in general and offers new solutions for therapeutic resolution of antibiotic resistant bacteria strains. While radioisotope therapy can be used to treat even the most dangerous forms of antimicrobial-resistant bacterial infections with limited side effects, target specificity remains lacking absent Applicant's novel claimed invention. By linking novel aptamers targeting specific bacteria, new therapeutic and diagnostic modalities are enabled. When used according to the claimed system, disclosed radioisotopes, which are very powerful, have multiple energies that are available for both therapeutic and diagnostic use. By utilizing highly specific novel aptamers binding to surface proteins of bacteria together with radioisotopes, the claimed aptamer radioisotope conjugate (701) kills bacterial targets with minimal side effects to healthy tissue without creating the increased risk of antibiotic resistance.

### REFERENCE TO AN ELECTRONIC SEQUENCE LISTING

The contents of the electronic sequence listing (EN8.xml; Size; 2,908 bytes; and Date of Creation: May 2, 2023 is herein incorporated by reference in its entirety.

### TECHNICAL FIELD

The claimed invention relates to novel anti-infective therapeutic compounds and methods. With greater particularity, the claimed invention discloses aptamer-radioisotope therapeutic agents used against microbiological infections. With still greater particularity, the claimed invention specifically targets S. aureus and E. coli infections with different energy levels of radioisotopes.

### BACKGROUND ART

Antimicrobial resistance is currently recognized as one of the greatest threats to human health worldwide. According to the World Health Organization (WHO), antimicrobial-resistant strains are present in all parts of the world, and new resistance mechanisms continue to emerge and spread globally. Rising rates of AMR will make it increasingly difficult and expensive to control and treat infections and could affect the sustainability of some modern healthcare interventions.

In the United States alone, at least 2 million people become infected with antibiotic-resistant bacteria, with approximately 23,000 associated deaths each year. Infection with these microorganisms is further complicated by the limited number of effective therapeutic options.

Without proper handling, it is projected that there will be 10,000,000 deaths by 2050 attributed to antimicrobial resistant infections. Therefore, a new approach to treating these infections is highly needed.

### SUMMARY OF INVENTION

### Technical Problem

Current therapeutics for microbial infections are rapidly losing their efficacy owing to anti-microbial drug resistance. A key problem with over administration of antibiotics is the ability of bacteria to adapt and become resistant to chemical antibiotics resulting in greatly diminishing or even no effect against bacterial replication.

New strategies against bacterial infection require not just new chemical entities, but entirely new anti-bacterial solutions.

### Solution to Problem

By linking novel aptamers targeting specific bacteria to radioisotopes using a linker, new therapeutic and diagnostic modalities are enabled. While radioisotope therapy can be used to treat even the most dangerous forms of antimicrobial-resistant bacterial infections with limited side effects, target specificity remains lacking absent Applicant's novel claimed aptamer linked conjugate.

When used according to the claimed system, disclosed radioisotopes, which are very powerful, have multiple energies that are available for both therapeutic and diagnostic use. By utilizing highly specific novel aptamers binding to surface proteins of bacteria together with radioisotopes, the claimed invention kills bacterial targets with minimal side effects to healthy tissue without creating the increased risk of antibiotic resistance.

### Advantageous Effects of Invention

The claimed invention provides a novel system and related method for treatment of bacterial infections in general and offers new solutions for therapeutic resolution of antibiotic resistant bacteria strains.

Moreover, radiotherapeutics are unlike chemical compounds in that increased resistance to treatment should not be a consequence with the claimed system and method.

By combining the novel S. aureus and E. coli aptamers providing infection target specificity conjugated with therapeutic radioisotopes, a novel, broad and robust anti-microbial health and wellness system is placed in the hands of patients and medical health care providers.

### BRIEF DESCRIPTION OF DRAWINGS

The accompanying drawings are included to better illustrate exemplary embodiments of the claimed invention.

FIG. 1 is a graphical chart illustration of aptamer affinity against E. coli.

FIG. 2 is a graphical chart illustration of aptamer affinity against S. aureus.

FIG. 3 is a schematic illustration of aptamer-radioisotope creation.

FIG. 4 is a graphical illustration which depicts the stability of EDTA, DTPA, and DOTA in human serum, RPMI medium and ammonium acetate.

FIG. 5 is a graphical illustration of DOTA in a twisted square antiprismatic coordination.

FIG. 6 is a schematic illustration of the manufacture of in a twisted square antiprismatic coordination.

FIG. 7 is a schematic illustration of aptamer-radioisotope composition.

FIG. 8 is a graphical illustration of DOTA-NHS.

FIG. 9 is a schematic illustration of DOTA-NHS combination with the antisense oligonucleotide (ASON) sequence of the aptamer to yield the conjugate according to the claimed invention.

FIG. 10 is a schematic illustration of conjugate radiolabelling oxidation.

FIG. 11 is a flowchart illustrating a preferred embodiment of the claimed invention.

FIG. 12 is a flowchart illustrating a preferred embodiment of the claimed invention.

### DESCRIPTION OF EMBODIMENTS

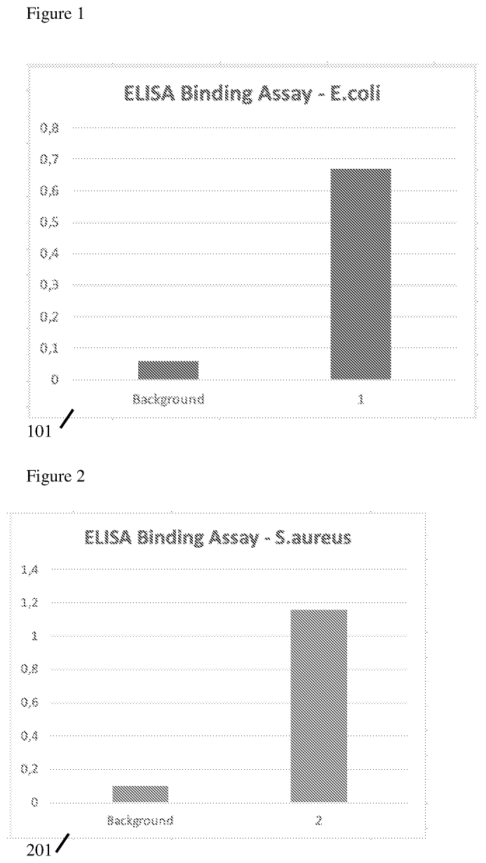

Examples: Example 1 Novel Engineered Aptamer for E. coli. Aptamers are short single stranded DNA/RNA molecules that can selectively bind to a specific target. The claimed invention utilizes Applicant's novel engineered aptamers isolated from E. coli in the first illustrative embodiment and S. aureus in the second illustrative embodiment that have specific binding towards two types of bacteria, E. coli and S. aureus. In the first illustrative embodiment, the affinity of the engineered aptamer for E. coli targets is demonstrated. FIG. 1 is a graphical chart illustration (101) of aptamer affinity against E. coli as measured by ELISA. In the first illustrative embodiment, as can be seen from FIG. 1, the binding of the first aptamer to E. coli is very high compared to its binding to the background (healthy cell).

Example 2: Novel Engineered Aptamer for S. aureus. In the second illustrative embodiment, the affinity of the engineered aptamer for S. aureus targets is demonstrated. FIG. 2 is a graphical chart illustration (201) of aptamer affinity against S. aureus as measured by ELISA. In the second illustrative embodiment, as can be seen from FIG. 2, the binding of the first aptamer to S. aureus is very high compared to its binding to the background (healthy cell). The fairly low binding of the aptamer to the background (healthy cell) demonstrates that the aptamer has good binding strength to the target (pathogen) and is highly specific. The specificity of the disclosed aptamer enables high target affinity which enables the killing of specific pathogens with minimal side effects.

The fairly low binding of the aptamer to the background (healthy cell) demonstrates that the aptamer has good binding strength to the target (pathogen) and is highly specific. The specificity of the disclosed aptamer enables high target affinity which enables the killing of specific pathogens with minimal side effects.

TABLE
Sequences of the aptamers are shown below:

| | | |
|---|---|---|
| 1 | TCCGGGAGGGGGGGTGGGTGGACGG | Target E. coli |
| 2 | GGTGGTGGCGGGGGGTGGGGGGGTT | Target S. aureus |

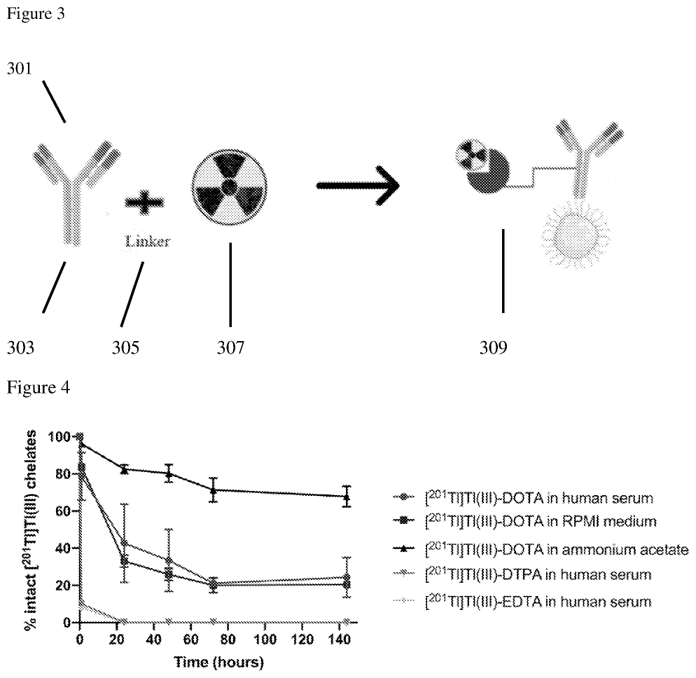

Radioisotopes: To complement the specific aptamer affinity for individual bacterial strains, aptamers are linked to radioisotopes for novel anti-bacterial effects. FIG. 3 is a schematic illustration (301) of aptamer-radioisotope (309) creation. Radioisotopes (307) are unstable forms of a chemical element that releases radiation as it breaks down and becomes more stable. The ionizing radiation emitted by these radioisotopes is targeted to bacteria in order to kill them. To create the aptamer-radioisotope construct (309), the aptamer of choice with specificity for a particular biological target (303) is joined by a linker (305) to the radioisotope (307). By specifically attaching to the target bacteria, the aptamer (303) will guide the radioisotopes (307) and help them kill the target with high-energy radiation. This will effectively kill the target with good outcome and few side effects. Additionally, the claimed invention is killing the bacteria using radiation, the chances of developing resistance are lower compared to conventional antibiotics.

Examples of linker (301) include DOTA, ethylenediaminetetraacetic acid (EDTA), and diethylenetriaminepentaacetic acid (DTPA). While all the chelators complexed thallium rapidly and efficiently, DOTA performed better than both EDTA and DTPA. This is shown in the FIG. 4, which depicts the stability of EDTA, DTPA, and DOTA in human serum, RPMI medium and ammonium acetate. After 1 hour of incubation in the human serum, 78±12% of the [201TI]TI(III)-DOTA complex still remained, whereas only 9±2% of the [201TI]TI(III)-DTPA and [201TI]TI(III)-EDTA complexes remained. After 144 hours, [201TI]TI(III)-DOTA was also still intact while the other complexes has completely dissociated by 24 hours. Similarly, [201TI]TI(III)-DOTA appeared relatively stable in RPMI medium and ammonium acetate, with 20±2% and 68±6% of the complex still remaining after 144 hours of incubation.

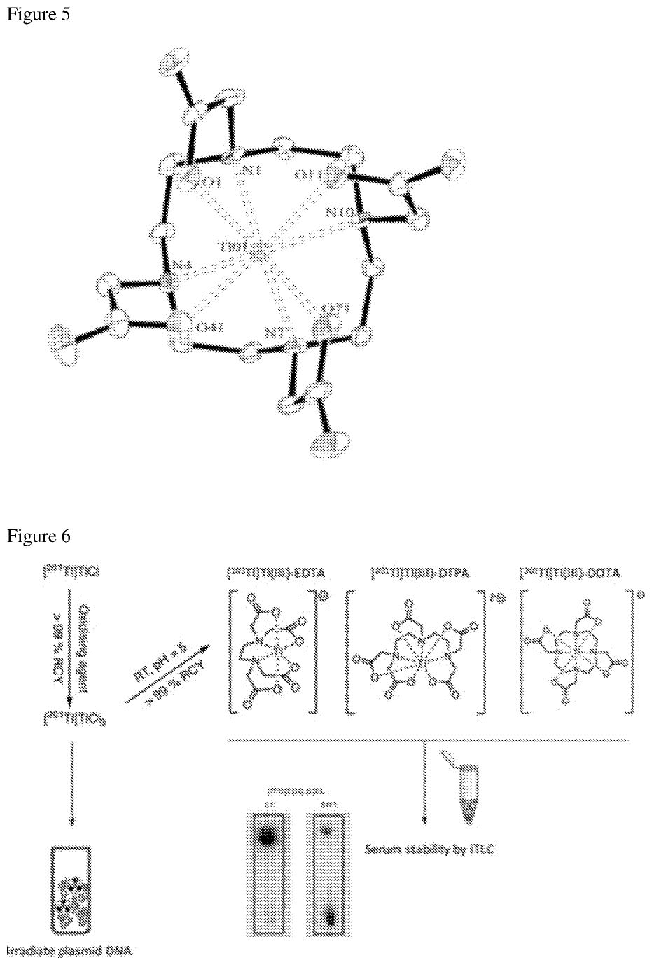

Due to EDTA and DTPA being acyclic chelators, having 6 and 8 donor atoms respectively, they are unstable and this results in their complexes being thermodynamically favorable but not kinetically stable. DOTA, on the other hand, enables a more stable chelation of thallium than DTPA, at least in vitro as the thallium ion directly coordinated to all the 8 donor atoms in a twisted square antiprismatic coordination as can be seen in the FIGS. 5 and 6. FIG. 5 is a graphical illustration of DOTA in a twisted square antiprismatic coordination and FIG. 6 is a schematic illustration of the manufacture of in a twisted square antiprismatic coordination. From all those conventional bifunctional chelators for Thallium (III), DOTA serves as the most promising for future molecular radionuclide therapy as it has greater kinetic stability compared to EDTA AND DTPA, which have inadequate and inconsistent stability.

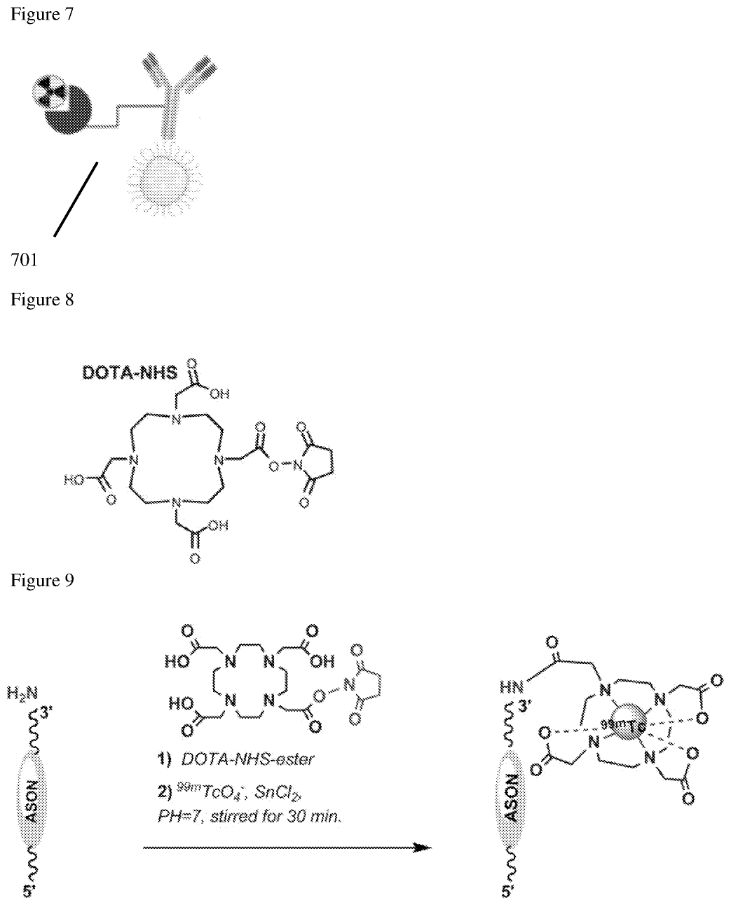

FIG. 7 is a schematic illustration of aptamer-radioisotope composition. The aptamer-radioisotope construct (701) according to the claimed invention includes a micro-organism specific aptamer joined by the linker to a desired radioactive isotope. Specific aspects of the radioisotope to linker to aptamer conjugate include conjugating the aptamer and linker first, then to the radioisotope. Foremost, the antisense oligonucleotide (ASON) sequence of the aptamer will be conjugated with DOTA-NHS via the amine-functionalization of the oligonucleotides of the 3′-end. To illustrate this relationship, FIG. 8 is a graphical illustration of DOTA-NHS and FIG. 9 is a schematic illustration of DOTA-NHS combination with the antisense oligonucleotide (ASON) sequence of the aptamer to yield the conjugate according to the claimed invention.

In a preferred embodiment of the claimed invention, DOTA-NHS dissolved in dry DMSO (3.85 μL) is added to a solution of the aptamer with a mixture containing equal volumes of sodium phosphate buffer (0.1M, pH 8.5, 200 μL) and sodium carbonate (0,1M, pH 8.5, 200 μL). This reaction mixture is stirred in the dark at room temperature for 2 hours and then desalted using a spin-filter dialysis using a 10 kDa molecular weight cut-off centrifugal filter to stop the reaction by buffer exchange with ultrapure water. Afterwards, the solution is purified by peak collection using semi-preparatory HPLC system. Collected fractions can then be frozen at −80° C., lyophilized overnight, dissolved in ultrapure water and stored at −20° C. or −80° C.
A few conditions are required to ensure a successful conjugation:
   50 equiv excess of the bifunctional chelator is required to achieve the goal of >98% desired product without the need for additional purification to remove excess reagents
   Occur at a mildly basic pH range (8-9) and conduct under aqueous conditions (0.1M NaHCO₃)
   Reactions are undertaken at low-temperature (4° C.) to produce high yield.
      Efficient peak collection during HPLC semi-preparative purification is also critical to achieve high chemical yields.

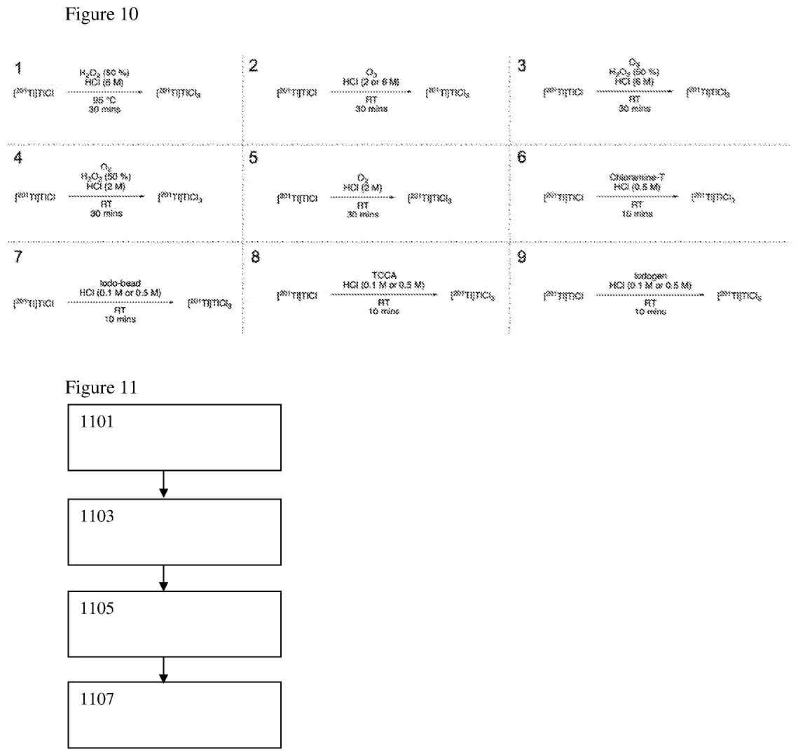

Following the binding of the linker and aptamer, the conjugate can then be radiolabelled by adding [²⁰¹TI]TICl₃ (40 μL, 3 MBq) to the conjugate solution in an Eppendorf tube with ammonium acetate buffer (0.25 M, pH 5, 100 μL). To obtain [201TI]TICl₃, [₂₀₁TI]TI⁺ must first be oxidized to [²⁰¹TI]TI³⁺, which can be done in a few methods shown in FIG. 10.

Radioisotope variations: Further embodiments of the claimed invention include five specific types of radioisotopes, which are categorized as Alpha, Auger, Beta, Gamma and Positron. Alpha, Auger and Beta are higher energy radioisotopes compared to Gamma and Positron, which consequently makes them better and more effective for therapeutic use. In the case of bacterial infection, however, gamma and positron can also be utilized therapeutically as the amount of energy required to kill bacteria are not as high as opposed to other diseases such as treating cancer.

Therapeutic Use: According to the claimed invention, a good range of radioisotopes with different energies are hereby provided. In terms of anti-microbial embodiments, there are various options available to treat different severity of infections. Gamma/positron can be first applied to see whether the radioisotope is strong enough to kill the target. If not, stronger energy radioisotopes such as alpha, auger or beta may be subsequently deployed. Some examples of radioisotopes according to the claimed invention are Thallium-201 and Copper-64.

Thallium-201 is an isotope of Thallium that is widely used for medical imaging, such as myocardial imaging, tumor diagnosis, coronary artery diagnosis, etc. It has been used for imaging heart function under stress and rest conditions since about 1975, which makes it one of the oldest and best studied of the present-day agents.

Cu-64 is a positron and beta emitting isotope of copper that have been previously used for molecular radiotherapy and positron emission tomography (PET). As a positron emitting isotope, which is generally used for diagnostic purposes such as imaging, the 12.7 hours half-life of Cu-64 provides the flexibility to image both smaller molecules and larger, slower clearing nanoparticles.

Diagnostic Use: when radioisotopes are used for diagnostic tests, it is desirable to reduce to a minimum the radiation dose delivered to the patient during the test. As gamma and positron have lower energy, they can also be used for diagnostic purposes. Before treatment, we can use the aptamer+radioisotope (gamma/positron) conjugate to diagnose the patient (to check the location, severity of infection, etc.). After diagnosis, we can follow up with treatment using stronger radioisotopes (alpha/auger/beta).

TABLE 2
Current radioisotope examples and functions

| | Radioisotope | | |
|---|---|---|---|
| Type | example | Function | Advantage |
| Alpha | Ac-225 | Therapeutic | High energy |
| | | | radioisotope that is |
| | | | highly effective for |
| | | | killing bacteria/fungus |
| Auger | In-111 | Therapeutic | High energy |
| | | | radioisotope that is |
| | | | highly effective for |
| | | | killing bacteria/fungus |
| Beta | Lu177 | Therapeutic | High energy |
| | | | radioisotope that has |
| | | | higher penetration power |
| Positron | F-18 (FDG) | Diagnostic + | Lower energy |
| | | Therapeutic | radioisotope that can |
| | | | be used for diagnostics, |
| | | | providing precise and |
| | | | clear imaging, and |
| | | | also can be used |
| | | | therapeutically to |
| | | | kill bacteria/fungus |
| Gamma | 99m-Tc | Diagnostic + | Lower energy |
| | (Technetium) | Therapeutic | radioisotope that can |
| | | | be used for diagnostics, |
| | | | providing precise and |
| | | | clear imaging, and |
| | | | also can be used |
| | | | therapeutically to |
| | | | kill bacteria/fungus |

Radiopharmaceutical Applications: With the prolonged usage of antibiotics, they are becoming less and less effective for treatment due to increasing bacteria resistance. As a result, it would require the use of second- and third-line treatments which can seriously harm patients; while some have no treatment options at all. On the other hand, radioisotope therapy can be used to treat even the most dangerous forms of antimicrobial-resistant bacterial infections with limited side effects. Thus, according to the claimed invention, radioisotopes are combined with novel aptamers to produce a drug with high specificity and energy to kill the target bacteria, for both therapeutic and diagnostic uses.

Advantages of the presently claimed system: When used according to the claimed system, disclosed radioisotopes, which are very powerful, have multiple energies that are available for both therapeutic and diagnostic use. Their escalating energy levels allow them to be utilized for adaptive radiotherapy for different severities of infections. Moreover, the claimed embodiments usage of radiation, which is harder to develop resistance to, to kill the bacteria minimizes the probability of resistance. This, combined with multiple disclosed embodiments of radioisotopes that allows different options for treatment in case pathogens develop resistance, can provide effective treatments for the ever-increasing antimicrobial resistance. With the highly specific binding to surface proteins of bacteria, the claimed invention also kills targets with minimal side effects to our body/our healthy cells.

FIG. 11 is a flowchart illustrating a preferred embodiment of the claimed invention for the method of treatment of a patient in need thereof with a therapeutic aptamer-radioisotope conjugate. The method begins with:
Preparing (1101) therapeutic aptamer-radioisotope conjugate by linking aptamer to radioisotope, optionally Diagnosing (1103) an alternate aptamer-radioisotope conjugate to determine infection levels
Administering (1105) therapeutic aptamer-radioisotope conjugate to a patient in need thereof, and
Confirming (1107) clearance of microbial infection.

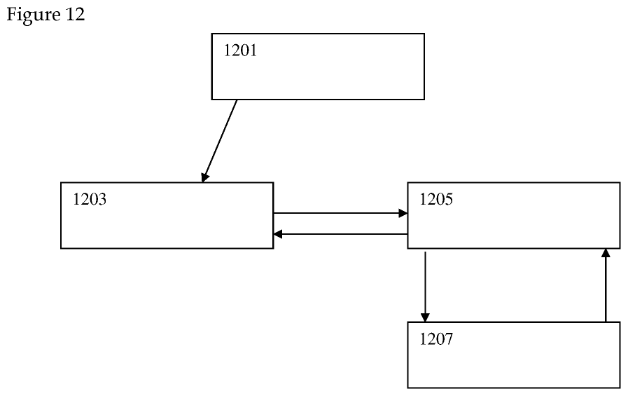

FIG. 12 is a flowchart illustrating a preferred alternate embodiment of the claimed invention. In the alternate embodiment,
Selecting (1201) aptamer based on anticipated microbial infection,
Determining (1203) radioisotope based upon anticipated microbial infection,
Administering (1205) therapeutic aptamer-isotope conjugate, and
Diagnosing (1207) with alternate aptamer-radioisotope conjugate to subsequently evaluate infection levels.

In the description, numerous specific details are set forth in order to provide a thorough understanding of the present embodiments. It will be apparent, however, to one having ordinary skill in the art that the specific detail need not be employed to practice the present embodiments. In other instances, well-known materials or methods have not been described in detail in order to avoid obscuring the present embodiments.

Reference throughout this specification to “one embodiment”, “an embodiment”, “one example” or “an example” means that a particular feature, structure or characteristic described in connection with the embodiment or example is included in at least one embodiment of the present embodiments. Thus, appearances of the phrases “in one embodiment”, “in an embodiment”, “one example” or “an example” in various places throughout this specification are not necessarily all referring to the same embodiment or example. Furthermore, the particular features, structures or characteristics may be combined in any suitable combinations and/or sub-combinations in one or more embodiments or examples. In addition, it is appreciated that the figures provided herewith are for explanation purposes to persons ordinarily skilled in the art and that the drawings are not necessarily drawn to scale.

As used herein, the terms “comprises,” “comprising,” “includes,” “including,” “has,” “having,” or any other variation thereof, are intended to cover a non-exclusive inclusion. For example, a process, article, or apparatus that comprises a list of elements is not necessarily limited to only those elements but may include other elements not expressly listed or inherent to such process, article, or apparatus. Additionally, any examples or illustrations given herein are not to be regarded in any way as restrictions on, limits to, or express definitions of any term or terms with which they are utilized. Instead, these examples or illustrations are to be regarded as being described with respect to one particular embodiment and as being illustrative only. Those of ordinary skill in the art will appreciate that any term or terms with which these examples or illustrations are utilized will encompass other embodiments which may or may not be given therewith or elsewhere in the specification and all such embodiments are intended to be included within the scope of that term or terms. Language designating such nonlimiting examples and illustrations includes, but is not limited to: “for example,” “for instance,” “e.g.,” and “in one embodiment.”

### INDUSTRIAL APPLICABILITY

The claimed invention has industrial applicability in the biomedical arts. In particular, the claimed invention is directly relevant to cardiac health and related therapeutic administration of pharmaceuticals for mitigation of and therapeutic effects against cardiac diseases.

### CITATION LIST

### Patent Literature

This patent application is a continuation-in-part and claims priority to U.S. patent application Ser. No. 16/374,838 filed Apr. 4, 2019 to Patrick Shau-park Leung entitled “Personalized Healthcare P4 Alzheimer's Detection System and Method” which claims priority to provisional patent application 62/653,547 filed Apr. 5, 2018. Furthermore this patent application is a continuation-in-part and claims priority to U.S. patent application Ser. No. 15/666,699 filed Aug. 2, 2017 to Patrick Shau-park Leung entitled “Personalized Glucose and Insulin Monitoring System.” In addition, this patent application is a continuation-in-part and claims priority to U.S. patent application Ser. No. 15/469,138 filed Mar. 24, 2017 to Patrick Shau-park Leung entitled “Public personalized mobile health sensing system, method and device” which is a continuation of U.S. patent application Ser. No. 15/056,163 filed Feb. 29, 2016 to Patrick Shau-park Leung entitled “Mobile automated health sensing system, method and device”.

### SEQUENCE LISTING

The contents of the sequence listing submitted in XML, format identified as “EN8.XML” created on 5/2/2023 and is 2,908 bytes in length is herein incorporated by reference in its entirety.
SEQ ID NO: 1
Length: 25
Type: DNA/RNA
Organism: Artificial Sequence
Description of Artificial Sequence: Synthetic oligonucleotide

SEQ ID NO: 2
5′-TCC GGG AGG GGG GGT GGG TGG ACG G-3′

Length: 25
Type: DNA/RNA
Organism: Artificial Sequence
Description of Artificial Sequence: Synthetic oligonucleotide

5′-GGT GGT GGC GGG GGG TGG GGG GGT T-3′

I claim:

1. A therapeutic aptamer-radioisotope conjugate system comprising:
Microbial specific aptamer operably linked to radioisotope.

2. The therapeutic aptamer-radioisotope conjugate system of claim 1 wherein said therapeutic aptamer-radioisotope conjugate system additionally comprises bacteria specific aptamers including aptamers corresponding to Seq ID #1 with the sequence of 5′-TCC GGG AGG GGG GGT GGG TGG ACG G-3′.

3. The therapeutic aptamer-radioisotope conjugate system of claim 1 wherein said therapeutic aptamer-radioisotope conjugate system additionally comprises bacteria specific aptamers including aptamers corresponding to Seq ID #2 with the sequence of 5′-GGT GGT GGC GGG GGG TGG GGG GGT T-3′.

4. The therapeutic aptamer-radioisotope conjugate system of claim 1 wherein the radioisotope is Ac-225.

5. The therapeutic aptamer-radioisotope conjugate system of claim 1 wherein the radioisotope is In-111.

6. The therapeutic aptamer-radioisotope conjugate system of claim 1 wherein the radioisotope is Lu177.

7. The therapeutic aptamer-radioisotope conjugate system of claim 1 wherein the radioisotope F-18 (FDG).

8. The therapeutic aptamer-radioisotope conjugate system of claim 1 wherein the radioisotope is 99m-Tc (Technetium).

9. The therapeutic aptamer-radioisotope conjugate system of claim 1 wherein the radioisotope is selected from the group consisting of Thallium-201 and Copper-64.

10. A method for therapeutic treatment of microbial infection comprising the steps of:
Preparing Aptamer-radioisotope conjugate by linking aptamer to radioisotope, Administering therapeutic aptamer-radioisotope conjugate to a patient in need thereof, and Confirming clearance of microbial infection.

11. The method for therapeutic treatment of microbial infection of claim 10 additionally comprising diagnosing infection utilizing a diagnostic strength radioisotope prior to administering therapeutic aptamer-radioisotope conjugate.
